(19) United States
(12) Patent Application Publication
(10) Pub. No.: US 2021/0322463 A1
(43) Pub. Date: Oct. 21, 2021

(54) METHOD FOR ACCELERATED TISSUE PENETRATION OF COMPOUNDS INTO BRAIN

(71) Applicant: LikeMinds, Inc., Boston, MA (US)

(72) Inventors: Kenneth L. RICE, Auburndale, MA (US); Peter G. SAVAS, Boston, MA (US)

(21) Appl. No.: 17/260,617

(22) PCT Filed: Jul. 18, 2019
(86) PCT No.: PCT/US2019/042331

§ 371 Date: Jan. 15, 2021

Related U.S. Application Data
(60) Provisional application No. 62/699,798, filed on Jul. 18, 2018.

(51) Int. Cl. A61K 33/00; A61K 9/00; A61K 51/04; A61K 31/46; A61P 25/16

(52) U.S. Cl. CPC A61K 33/00; A61K 9/007; A61P 25/16; A61K 9/0019; A61K 31/46; A61K 51/0448

(57) ABSTRACT

Disclosed is a method of increasing the rate of delivery of an active compound or agent to the brain across the blood brain barrier of a subject. In one aspect, the disclosure provides an accelerated method of providing an active compound to a targeted region of the brain of a subject, served by the cerebral medial artery, the method comprising: administering a sub-anesthetic dose of xenon as an inhalant to the subject; and administering an efficacious amount of the active compound to the bloodstream of the subject, the active compound reaching the targeted region of the brain in less time relative to a time it takes to reach the targeted region without xenon administration.

### CROSS-REFERENCE TO RELATED APPLICATIONS

This application claims priority to U.S. provisional application No. 62/699,798, filed Jul. 18, 2018, the contents of which is hereby incorporated in its entirety.

### FIELD OF THE INVENTION

The present disclosure relates to human and veterinary medicine. More specifically, the disclosure relates methods of treating brain disorders and of diagnosing and monitoring the treatment of certain brain disorders.

### BACKGROUND

Many diagnostic and therapeutic interventions in brain disease depend on the rapid transition of molecules designed for diagnosis or treatment of brain diseases from the vasculature into the brain tissue where the targets for diagnostic and/or therapeutic intervention are located. These targets can be transporter systems, synapses, receptors, and other structures located on or in the cell membranes or within the cytoplasm of cerebral cells as well as compounds deposited in the interstitium of the brain tissue. Sufficient cerebral blood flow (CBF) is essential for delivering appropriate amounts of blood to the brain tissue.

For example, in imaging procedures, payloads of imaging compounds need to be delivered across the blood brain barrier and need to transition from the intravascular space to the interstitial space to reach targets of interest. Fast transition to and from the vascular space into the interstitial space is important especially if the payload contains a radiopharmaceutical intended to saturate available receptors within a given useful time span during which scanning of signals takes place.

To calculate actual blood flow through a vessel, the measurement of blood velocity and vessel diameter are important. If the diameter of the vessel is assumed to be constant, a change in blood velocity will proportionally reflect increased flow through the vessel and at equilibrium will be accompanied by increased flow in the distal territory supplied by that vessel. During distal vasodilation or vasoconstriction, distal blood flow transiently may be greater or less than flow in the parent artery, but at equilibrium, total flow in the parent vessel and its distal circulation must be the same. Increased MCA blood velocity accompanied by, and slightly preceded by, a decrease in the Gosling pulsatility index, suggests that the increase in velocity may be due to decreased distal vascular resistance rather than to changes in the diameter of the MCA or in blood pressure (Giller et al. (1990) J. Neurosurg. 72(6) 901-906).

Maintaining or achieving normal blood flow to the affected region (s) of the brain is important for the treatment of many brain disorders. For example, movement disorders like Parkinson Disease (PD) are characterized by dysfunction of dopaminergic neurons, predominantly located in the substantia nigra of the basal ganglia, located within the supply region for the MCA. In the case of PD, variances in regional CBF seem to be associated with clinical symptoms of the disease (see, e.g., Aiba et al. (1994) Rinsho Shinkeigaku 34(11): 1099-104)). Thus, normal CBF is desirable for proper function of brain tissue.

Accordingly, what is needed are methods which improve CBF in order to provide enhanced delivery of medicaments and imaging agents to the brain. Also what is needed are improved methods of imaging, and monitoring the treatment of, brain disorders in an affected subject.

### SUMMARY

It has been discovered that when CBF in a subject is increased following xenon gas inhalation, immediately thereafter, resistance vessels in the brain undergo vasodilation, and the resulting increase in regional CBF particularly to the basal ganglia. This increase in blood supply to the basal ganglia and associated structures enables the selective targeting of therapeutic and diagnostic imaging agents to those regions.

This discovery has been exploited to provide the present invention, which, in part, is directed to methods for the accelerated and targeted delivery of diagnostic and/or therapeutic compounds to a targeted region of the brain.

In one aspect, the disclosure provides an accelerated method of providing an active compound to a targeted region of the brain of a subject, served by the cerebral medial artery, the method comprising: administering a sub-anesthetic dose of xenon as an inhalant to the subject; and administering an efficacious amount of the active compound to the bloodstream of the subject, the active compound reaching the targeted region of the brain in less time relative to a time it takes to reach the targeted region without xenon administration.

In some embodiments, the sub-anesthetic dose of xenon is from about 10% to about 43% xenon gas. In particular embodiments, xenon is administered to the subject before, concomitantly with, or after the administration of the active compound.

In some embodiments, the subject suffers from a disorder of the basal ganglia in the vascular bed of the middle cerebral artery. In certain embodiments, the disorder is a dopaminergic-related disorder.

In some embodiments, the active compound is administered intravenously to the patent. In certain embodiments, the active compound is an imaging or diagnostic agent. In particular embodiments, the diagnostic agent is an imaging agent for SPECT, PET, or MRI. In specific embodiments, the imaging agent is DaTScan, DaT2020.

In some embodiments, the active compound is a therapeutic compound for treating a dopaminergic disorder such as, but not limited to, Parkinson's disease, attention deficit hyperactivity disorder, dementia, clinical depression, anxiety, narcolepsy, obesity, sexual dysfunction, schizophrenia, bipolar disorder, nausea/vomiting, addictive disorder (drugs, smoking), pheochromaocytoma, or binge eating disorder.

In another aspect, the disclosure provides an improved method of evaluating an ability of a therapeutic composition to modulate a dopaminergic activity in the brain of a subject suspected of having a dopaminergic disorder, the improvement resulting in an accelerated delivery of the therapeutic composition across the blood brain barrier to a region of the brain served by the cerebral medial artery. The improvement comprises administering to the subject a sub-anesthetic amount of xenon gas as an inhalant at about 10% to about 43% before or during administering radiolabeled tropane to the subject; determining a baseline level and pattern of binding of the administered radiolabeled tropane to dopaminergic neurons in a portion of the brain of the subject; treating the subject with an initial dose of a first therapeutic composition; administering radiolabeled tropane to the treated subject; and determining the level and pattern of radiolabeled tropane binding to the portion of the brain of the treated subject, a change in level and/or pattern of radiolabeled tropane binding in the treated subject relative to baseline levels and/or patterns of radiolabeled tropane binding being indicative of the ability of the therapeutic composition to modulate a dopaminergic activity in the brain,

In some embodiments, the dopaminergic disorder is Parkinson's disease, attention deficit hyperactivity disorder, dementia, clinical depression, anxiety, narcolepsy, obesity, sexual dysfunction, schizophrenia, bipolar disorder, nausea/vomiting, addictive disorder (drugs, smoking), pheochromaocytoma, or binge eating disorder.

In certain embodiments, the tropane is radiolabeled with ¹²³I, ¹²⁴I, ¹²⁵I, ¹⁸F, ^{99m}Tc, or ¹¹C. In other embodiments, the tropane is radiolabeled with ¹²³I, ¹²⁴I, ¹²⁵I, or ^{99m}Tc, and the level and pattern of binding of radiolabeled tropane is measured by SPECT. In other embodiments, the tropane is radiolabeled with ¹⁸F, ¹²⁴I, and ¹¹C, and the level and pattern of binding of radiolabeled tropane is measured by PET.

In some embodiments, the method further comprises: treating the subject with a secondary dose of the therapeutic composition which is different from the initial treatment dose if the level of radiolabeled tropane binding is decreased; administering the radiolabeled tropane to the subject treated with the secondary dose; and determining the level and pattern of radiolabeled tropane binding, an increase in the level of radiolabeled tropane binding being indicative of the ability of the secondary dose of the therapeutic composition to positively modulate a dopaminergic activity in the brain.

In other embodiments, the method further comprises: treating the subject with a second therapeutic composition which is different from the first therapeutic composition; administering radiolabeled tropane to the subject treated with the second therapeutic composition; and determining the level and pattern of radiolabeled tropane binding, an increase in the level and/or pattern of radiolabeled tropane binding being indicative of the ability of the first and second therapeutic composition to modulate a dopaminergic activity in the brain.

In yet other embodiments, the method further comprises: treating the subject with the first therapeutic composition and a second therapeutic composition which is different from the first therapeutic composition; administering radiolabeled tropane to the subject treated with the first and second therapeutic compositions; and determining the level and pattern of radiolabeled tropane binding, an increase in the level and/or pattern of radiolabeled tropane binding being indicative of the ability of the first and second therapeutic compositions to modulate a dopaminergic activity in the brain.

In another aspect, the disclosure provides a method of treating a brain disorder (e.g., affecting the basal ganglia) in a subject, by increasing the blood supply to that region by inhalation of sub-anesthetic amounts of xenon gas as described above, in combination with a therapeutic agent. The xenon can be administered with, before, or after the administration of the therapeutic agent.

The present disclosure also provides a method of obtaining a time course for progression of a neurological disorder. The method includes treatment with a sub-anesthetic dose of xenon as described above, a detection process and a subsequent process. The detection process includes administering a radioactively labeled tracer molecule to a subject; and acquiring a first tomographic image of the subject. The subsequent process includes repeating the detection process once at a first subsequent time period to obtain a second tomographic image of the subject.

In an embodiment, the subsequent process includes repeating the detection process twice, once at a first subsequent time period and once more at a second subsequent time period that is after the first subsequent time period, to obtain a second tomographic image during the first subsequent time period and a third tomographic image during the second subsequent time period. In another embodiment, the subsequent process includes repeating the detection process multiple times.

In certain embodiments, the tracer is 2-carbomethoxy-3-(4-fluorophenyl)-N-(1-haloprop-1-en-3-yl) nortropane. In a particular embodiment, the tracer is 2-carbomethoxy-3-(4-fluorophenyl)-N-(1-iodoprop-1-en-3-yl) nortropane. The iodine of the tracer can be ¹²³I. The acquiring step can be performed via a SPECT scanner. Alternatively, the iodine of the tracer can be ¹²⁴I, in which case the acquiring step can be performed via a SPECT scanner or a PET scanner.

In another aspect, the disclosure provides a method of optimizing a treatment regimen. The method includes administering to a subject treated with a sub-anesthetic dosage of xenon as described above and a therapeutic agent an amount of a radiolabeled tracer molecule; determining a level of bound tracer by scanning the subject with tomography; and changing the agent or its dosage based on the level of the bound tracer. The xenon can be administered with, before, or after the administration of the therapeutic agent and/or the tracer.

In one embodiment, the tracer is 2-carbomethoxy-3-(4-fluorophenyl)-N-(1-iodoprop-1-en-3-yl) nortropane. In some embodiments, the iodine of the tracer is selected from the group consisting of ¹²³I and ¹²⁴I. The tomography, in certain embodiments, is SPECT.

In yet another aspect, the disclosure provides a method of determining effectiveness of a neuroprotective agent. The method includes administering to a subject treated with the neuroprotective agent an amount of a radioactively labeled tracer molecule; acquiring a tomographic image of a portion of the subject via a method selected from the group consisting of SPECT and PET; determining a level of tracer binding from the tomographic image; and determining effectiveness of the neuroprotective agent based on the level of tracer binding as compared to a projected level, wherein the projected level is obtained from at least one previously obtained tomographic image. The method can include the administration of a sub-anesthetic dosage of xenon with, before, or after the administration of the neuroprotective agent.

In one embodiment, the projected level is obtained from one previously obtained tomographic image, and the projected level is equal to a previously obtained level of tracer binding from the previously obtained tomographic image. In other embodiments, the projected level is obtained from two or more previously obtained tomographic images, and the projected level is obtained through a regression analysis of levels found in the previously obtained tomographic images.

In certain embodiments, the tracer is 2-carbomethoxy-3-(4-fluorophenyl)-N-(1-iodoprop-1-en-3-yl) nortropane. The iodine of the tracer can be selected from the group consisting of ¹²³I and ¹²⁴I. The tomography can be SPECT, for example head-only SPECT.

In still another aspect, the disclosure provides a method of detecting binding of a radiolabeled tracer to dopamine transporter molecules. The method includes administering an amount of a radiolabeled tracer molecule to a subject; initiating a tomographic image acquisition about 15 minutes after administering the tracer; and terminating the tomographic image acquisition about 5 minutes to 10 minutes after initiating it if a tomographic image acquired until then reveals a pattern having two comma-shaped regions that are bilaterally symmetric with each other.

In some embodiments, the tracer is 2-carbomethoxy-3-(4-fluorophenyl)-N-(1-iodoprop-1-en-3-yl) nortropane. The iodine of the tracer can be ¹²³I or ¹²⁴I. The acquiring step can be performed via a SPECT scanner that scans only a portion of the subject, wherein the portion includes an area of the head of the subject.

The aspects and embodiments described above have various advantages. For example, they allow a clinician to obtain results in a quicker way as compared to the previously available methods. This is partially due to the increased sensitivity of the used tracer and to the tracer's faster passage from the blood-brain barrier. As a result, the methods are also more accurate than the previously available methods.

In addition, the disclosed methods avoid the problems caused by heterogeneous patient populations, concomitantly benefiting drug discovery efforts as well as accurate tuning of treatment regimens by providing an enhanced time course for disease progression. Because the methods can be employed even when the motor symptoms are not detectable, they also allow an earlier diagnosis of a disorder. Furthermore, due to the information-richness of the images, which provide not only a density or amount metric for DaT but also pattern information for DaT, the methods allow a clinician to more accurately distinguish between different disorders.

### DESCRIPTION

The disclosures of any patents, patent applications, and publications referred to herein are hereby incorporated by reference in their entireties into this application in order to more fully describe the state of the art as known to those skilled therein as of the date of the invention described and claimed herein. The instant disclosure will govern in the instance that there is any inconsistency between the patents, patent applications, and publications and this disclosure.

Unless defined otherwise, all technical and scientific terms used herein have the same meaning as commonly understood by one of ordinary skill in the art to which this disclosure belongs. The initial definition provided for a group or term herein applies to that group or term throughout the present specification individually or as part of another group, unless otherwise indicated.

The present disclosure provides methods of diagnosing, imaging, and treating certain brain disorders by increasing vessel dilation in the brain and increasing regional cerebral blood flow (rCBF) to the basal ganglia in the supply area of the Middle Cerebral Artery (MCA) following the inhalation of xenon gas at sub-anesthetic concentrations. This increase in rCBF results in an increased oxygen and nutrient supply to cells in the area of the basal ganglia, thereby leading to an improvement in certain brain disorders. This increase in CBF also resulting from xenon inhalation also provides methods of the accelerated delivery of therapeutic and imaging or monitoring agents to the brain. Faster delivery of therapeutic agents can result in faster resolution or improvement of the disorder. Accelerated delivery of monitoring or imaging agents results also in faster diagnosis of disease and in a faster determination if the therapeutic agent is efficacious.

### 1. Xenon

Xenon is a noble gas found in the earth's atmosphere. It is a commercially available gas used as an anesthetic as it interacts with multiple receptors and ion channels in the brain.

Xenon also induces neuroprotection through different mechanisms. It is an antagonist of the N-methyl-D-aspartate receptor. Once inhaled, xenon binds to [Nu PAR] and regulates calcium channels, thereby mitigating the negative effects of excessive calcium that leads to neuronal cell death in patients with compromised blood flow and poor oxygenation of brain tissue (NeurproteXenon). It also reduces the expression on genes associate with apoptosis and increases anti-apoptosis proteins after neuronal injury. In addition, it upregulates the reparative and restorative responses to oxygen deprivation, even under normoxic conditions. Also, xenon mimics neuronal ischemic preconditioning by activating ATP-sensitive potassium channels (Bantel et al. (2010) Anesthesiol. 112(3): 623-630).

In addition, xenon has been used widely for diagnostic purposes. Xenon-assisted SPECT, CT, and MRI techniques have been used for brain scans, mainly for imaging brain anatomy and function and the anatomy of vascular beds. For example, xenon-133 has been used as an inhaled radiopharmaceutical imaging agent to image and evaluate pulmonary function and to assess cerebral blood flow.

Naturally occurring xenon is composed of eight stable isotopes. However, the isotope composition administered may be different than that of natural xenon but must be of high medical purity. It can be provided as a compressed gas in containers such as compressed gas cylinders or cans. In the present disclosure, xenon is administered in a breathable gas with an amount of frim about 1% to about 40% by volume. The remaining gas is air or oxygen with other gases such as NO. or another inert gas. The gas may be administered using a ventilator having a gas-metering device or with an anesthesia machine. For example, the Calidose Dispenser system can be used to dispense Xenon-133 (Lantheus Medical Imaging, N. Billerica, Mass.).

### 2. Treatment of Brain Disorders with Xenon and Therapeutic Compounds

An increased rate of delivery of therapeutic, “active” compounds to areas of the brain served by the cerebral artery for the treatment of a neurological brain disease is achieved via their administration, during, and/or following and during xenon inhalation. This accelerated delivery of active compound results in the faster saturation of cellular and tissue targets for therapeutic purposes may allow for active compound dose reduction, duration, and/or d or an enhanced therapeutic effect.

For examples, xenon can be delivered before, during, or after administration of the active compound to the blood. If xenon delivery is supplied before, the period of time of xenon delivery ranges from about 1 minute to about 20 minutes before active compound administration. If delivered after, the xenon gas is delivered from about one minute to 10 minutes after administration of the active compound. Alternatively, the active compound and xenon are delivered simultaneously, and xenon or active compound administration can continue after that. If the compound is delivered as a bolus, xenon delivery can continue for up to 30 minutes.

Examples of useful active compounds include any efficacious drugs deliverable that are delivered to the blood and that can pass through the blood brain barrier without reducing their efficacy These include a variety of pharmacological agents which are neuroactive. As used herein, the term “neuroactive” encompasses compositions and drugs that are neuroprotective, disease-modifying, and/or symptom controlling with regard to neurological disorders. The term “neuroprotective” refers to that which serves to protect nerve cells against damage, degeneration, or impairment of function.

Non-limiting examples for neuroactive agents for PD include L-DOPA, bromocriptine, cabergoline, lisuride, pergolide, pramipexole, ropinirole, rotigotine, apomorphine, piribedil, rasagiline, and combinations thereof. Non-limiting exemplary neuroprotective agents for ADHD include amphetamine, dextroamphetamine, lisdexamfetamine, methamphetamine, methylphenidate, atomoxetine, clonidine, guanfacine, and combinations thereof. Non-limiting exemplary neuroactive agents for LBD include donepezil, rivastigmine, levodopa, melatonin, clonazepam, quetiapine, carbidopa-levodopa, and combinations thereof. Non-limiting exemplary neuroactive agents for clinical depression include fluoxetine, paroxetine, sertraline, citalopram, escitalopram, duloxetine, venlafaxine, desvenlafaxine, levomilnacipran, bupropion, trazodone, mirtazapine, vortioxetine, vilazodone, imipramine, nortriptyline, amitriptyline, doxepin, trimipramine, desipramine, protriptyline, tranylcypromine, phenelzine, isocarboxazid, selegiline, and combinations thereof Therapeutic agents that are not yet known but are being or will be developed can be assessed as well.

Other useful active compounds include those used to effectively treat a disease that is associated with decreased release of dopamine in the relevant brain structures, for example schizophrenia, are faced with numerous challenges. They must select a generally recommended therapy from a list of available first generation drugs such as, but not limited to: chlorpromazine (Thorazine) (Generic Only); fluphenazine (Generic) Prolixin (Brand) (Novartis, East Hanover, N.J. or Bristol-Myers Squibb, New York, N.Y.); haloperidol (Generic) Haldol (Brand) (Ortho McNeill Janssen Pharmaceuticals, Raritan, N.J.); loxapine (Generic) Loxitane (Brand) (Actavis, Plc, Parsippany-Troy Hills, N.J.); perphenazine (Generic) Trilafon (Brand) (Schering-Plough, Kenilworth, N.J.); thioridazine (Generic) Mellaril (Brand) (Novartis Pharmaceuticals, East Hanover, N.J.); thiothixene (Generic) Navane (Brand) (Pfizer, Inc., New York, N.Y.); trifluoperazine (Stelazine) (Generic Only).

These drugs are known as “neuroleptics” because, although effective in treating positive symptoms (acute symptoms such as hallucinations, delusions, thought disorder, loose associations, ambivalence, or emotional lability), they can cause cognitive dulling and involuntary movements, among other side effects. These medications are not so effective against “negative” symptoms such as apathy and decreased motivation. Newer drugs have been developed and launched which mitigate the negative side effects. Among these are: clozapine (Generic) Clozaril (Brand) (Novartis Pharmaceuticals, East Hanover, N.J.); aripiprazole (Generic) Abilify (Brand) (Otsuka Pharmaceutical Co. Ltd., Rockville, Md.); aripiprazole lauroxil (Generic) Aristada (Brand) (Alkermes Pharma, Athlone Co., Westmeath, Ireland); asenapine (Generic) Saphris (Brand) (Allergan, Plc, Westport Co., Mayo, Ireland); brexpiprazole (Generic) Rexulti (Brand) (Otsuka Pharmaceutical Co. Ltd., Rockville, Md.); cariprazine (Generic) Vraylar (Brand) Allergan, Plc, Westport Co. Mayo, Ireland); lurasidone (Generic) Latuda (Brand) (Sunovion, Marlborough, Mass.); paliperidone (Generic) Invega Sustenna/Invega Trinza (Brand) (Janssen Pharmaceutical, Raritan, NJ..); paliperidone palmitate (Generic) Invega Trinza (Brand) (Janssen Pharmaceutical, Raritan, N.J.); quetiapine (Generic) Seroquel (Brand) (Astra Zeneca, Cambridge, England); risperidone (Generic) Risperdal or Risperdal Consta (Brand) (Janssen Pharmaceutical, Raritan, N.J.); olanzapine (Generic) Zyprexa (Brand) (Eli Lilly and Co., Indianapolis, Ind.); ziprasidone (Generic) Geodon (Brand) (Pfizer, Inc. New York, N.Y.).

Generally applicable methods of preparing medicinal or pharmaceutical formulations of neuroactive agents are well known in the art. In addition, various aspects of preparing medicinal or pharmaceutical formulations as well as including additional components (e.g., stabilizers, antibacterials, antifungals) in these formulations are described in Remington: The Science and Practice of Pharmacy, ibid. and in Ansel's Pharmaceutical Dosage Forms and Drug Delivery Systems, ibid. By using these resources in addition to relying on what is commonly known in the art, a person of skill in the art would be able to prepare the disclosed formulations, modify them to better suit individual situations, add additional components, and optimize concentrations of various components.

Therapeutic agents are administered to a subject at the various doses known by those physicians and clinicians who care for patients with the disorder. The dosages of many therapeutic agents are known in the art (see, e.g., Allen (2013) Remington: The Science and Practice of Pharmacy (Pharmaceutical Press; London; 22nd ed.) as well as in Allen et al. (2001) Ansel's Pharmaceutical Dosage Forms and Drug Delivery Systems (Lippincott Williams & Wilkins; Philadelphia, 9th ed.)) are administered via a physician or clinician. The efficacy and effectiveness of each of these therapeutic agents is monitored.

For example, a neuroactive agent is administered at about 0.05 mg/day to about 250 mg/day, or about 0.5 mg/day to about 25 mg/day, or about 1 mg/day to about 4 mg/day. The administration may be oral. Alternative routes of administrations are possible as well (e.g., transdermal, subcutaneous, intravenous).

### 3. Diagnostic and Imaging Methods

The present disclosure also provides diagnostic and imaging methods which accelerate the rate of delivery of imaging agents for diagnosis of brain diseases and disorders at the cellular level or tissue level. Xenon gas inhalation is used to increase blood flow to the brain, and is used before, during, and/or after intravenous or systemic administration of the imaging agent.

If xenon delivery is supplied before, the period of time of xenon delivery ranges from about one minute to 10 minutes before imaging agent administration. If delivered after, the xenon gas is delivered from about 1 minute to 10 minutes after administration of the imaging agent. Alternatively, the imaging agent and the xenon gas are delivered simultaneously.

Note that when xenon is used, the dose of imaging agent that is useful may be reduced relative to the dose that is useful without xenon delivery. Due to faster saturation of cellular and tissue targets for diagnostic imaging, doses and/or exposure times can be shortened proportional to increase in rCBF. The dose refers to the amount of imaging agent delivered as a bolus or the length of time the agent needs to be administered.

Examples of useful imaging and diagnostic agents include, but are not limited to, DaTScan, DaT2020, derivatives thereof. Non-limiting examples of some useful SPECT-readable radiotracers for DaT detection include [¹²³I]-2β carbomethoxy-3β-(4-iodophenyl)tropane ([¹²³I]-beta-CIT); ^{[123}I]-2β-carbomethoxy-3β-(4-iodophenyl)-N-(3-fluoropropyl)nortropane ([¹²³I]-FP-CIT); ^{[123}I]-altropane; and [⁹⁹mTc]-TRODAT-1. Among these, [¹²³I]-FP-CIT is stable for 4 hours post-injection, has a half-life of about 13 hours, emits gamma rays with an energy of 159 keV, and is FDA approved. It can be administered at a dosage of 111 MBq-185 (185) MBq, and a scan dosage of 2.3 mSv to 4.4 mSv.

PET-readable radiotracers for DaT detection include [¹¹C]2-carbomethoxy-3-(4-¹⁸F-fluorophenyl)tropane ([¹¹C]CFT), [¹⁸F]CFT, ¹¹C-2β-Carbomethoxy-3β-(4-tolyl)tropane (¹¹C-RTI-32), [¹⁸F]-FP-CIT, and 11C-methylphenidate. PET can also be used to detect aromatic amino acid decarboxylase (AADC) by using ¹⁸F-3,4-dihydroxyphenylalanine (¹⁸F-DOPA), or vesicle monoamine transporter (VMAT2) by using [¹¹C]dihydrotetrabenazine or [¹⁸F]dihydrotetrabenazine.

In one nonlimiting example, a neuroprotective agent, or DaT2020, that can be administered to a subject, which subject can then be assayed for binding of DaT2020 to DaT molecules within about 15 minutes of administration. Because DaT2020 is labeled, the assay methods can employ SPECT, or PET, depending on the label used. In addition, the methods provide for monitoring of neuroprotective agent effectiveness though repeated scans.

### 4. Methods of Monitoring Drug Treatment

The present disclosure also provides improved companion methods of determining if the treatment being provided for a brain disorder has been or is efficacious. In these methods, after treatment with an active compound has occurred, imaging to measure the status of the disorder can be done.

When the concern is one particular neurodegenerative condition, changes in the imaged patterns and levels of tracer binding to the affected part of the brain indicates whether a certain pharmacologic or non-pharmacologic treatment regimen is efficacious. For example, a working drug may convert an asymmetric period-shaped pattern seen in PD into a symmetric comma-shaped pattern. For the state of the art, a working drug might preserve the pattern seen in PD along different image acquisitions at different time periods, in effect indicating that PD is no longer getting worse (i.e., it is maintained at a certain level, in other words the density of DaT molecules is not further decreasing due to the action of the drug). When monitoring the time course of a condition is of concern, imaging can provide information to ascertain whether the condition is being kept in check or whether it is further deteriorating.

As described above, xenon gas is delivered before, during and/or after active compound administration, and then again before, during. and/or after an imaging agent is administered.

### 5. Neurological Disorders to be Diagnosed/Treated

The provided methods can be used to assay for a variety of conditions, the treatment of which can then be monitored. Among these are the following: PD, ADHD, dementia, clinical depression, anxiety, narcolepsy, obesity, sexual dysfunction, schizophrenia, essential tremor, bipolar disorder, nausea/vomiting, addictive disorder (drugs, smoking), pheochromaocytoma, or binge eating disorder.

In particular, information regarding the time courses of PD, CBD, PSP, MSA, and LBD can be obtained by the present methods. For each, in some embodiments, the same average pattern from a representative sample of healthy subjects can be used as a control image. Representative averaged images of each can also be used to differentiate between these disorders. In addition, with a collection of images for one particular condition over a timeline, changes in both the overall levels of DaT binding and in the shapes of the patterns can be evaluated to assess the progression of the condition.

Reference will now be made to specific examples illustrating the disclosure. It is to be understood that the examples are provided to illustrate exemplary embodiments and that no limitation to the scope of the disclosure is intended thereby.

### EXAMPLES

### Example 1

### Combination Xenon—Active Compound Treatment

For studying a particular drug of concern (e.g., Azilect® (rasagiline)), first administer about 10% to about 30% xenon gas via inhalation for 10 minutes followed by administering [¹²³I]-2-carbomethoxy-3-(4-fluorophenyl)-N-(1-iodoprop-1-en-3-yl)nortropane (a tracer) at a dose of 3.5±1.0 mCi (296 MBq) [¹²³I]-DaT2020 (LikeMinds, Boston, Mass., followed by a 10 mL saline flush. to the subject, and by acquiring a SPECT tomograph of about 30 minutes in duration about 15 minutes after the administration. This serves as the first tomograph, from which it can be determined (1) a first density of DaT; (2) a first amount of DaT; and (3) a pattern of DaT. Each of these is informative, e.g., the pattern being comma shaped or not can indicate whether the subject has PD to begin with. The rest of the description will only refer to a level (“first level” for the first tomograph) of DaT from the tomograph. The term “level” encompasses the concepts of both the density and amount. Subsequently, the drug is administered to the subject. After a period of time post-drug administration, the tracer-administration and tomograph-acquisition steps can be repeated to obtain a second tomograph, which reveals a second level for DaT. Comparing the second level to the first level provides some indication as to if the drug is effective. For example, if the level has remained the same, this indicates that PD is at least not progressed and that the drug is efficacious. Subsequently, additional cycles of tracer-administration and tomograph-acquisition steps are performed, from which obtain additional tomographs are obtained, and thus, additional DaT levels. Ultimately, a time course for the progression of the disease is constructed, which provides data that not only more informative than what is obtained without recourse to a time course, but also is more objective than that obtained by subjective visual observations of a subject. Such a time course also enables comparisons of results across different research or clinical groups.

Creation of such time courses, which the disclosed methods enable, provides additional uses. For example, time courses for PD (or other neurological diseases) progression obtained from a number of individuals who have not been subjected to treatment. These time courses, or their various statistical averages, serve as useful controls for how progressive a disease is. A linear average value for the post-motor symptoms period slope of progression can be obtained, such as change in DaT levels divided by passed time. Alternatively, a hyperbolic, exponential, or multi-order polynomial model, can be fitted into the data to model it. Such models enable the following: (1) a prediction of how far the disease will have progressed at a certain time in the future; and (2) a forecasting of a projected DaT level for a certain time in the future, which would enable the testing of the effectiveness of a drug at that time point by comparing the DaT levels of a treated subject to those that are forecasted.

Effectively, a time course for disease progression is obtained both to assess drug effectiveness, and to gain information about the natural disease progression in the absence of any treatment. When used during a treatment regimen, a time course can be used to optimize the treatment regimen. For example, by comparing the projected values from the time course to those found from the tomograph of the treated subject, a decision to: (1) change the drug; (2) change the dosage; or (3) add or remove a drug from the regimen can be made.

### Example 2

### Assessment of Disease Modification

To determine if a therapeutic agent of interest is having the desired disease-slowing or disease-modifying effect on a patient, a combination of xenon delivery and DaT imaging can be used, xenon gas is administered via inhalation approximately one minute after injection of the DAT imaging agent at a concentration of 10% to 30% for a duration of 10 minutes. An initial (baseline) level and pattern of DaT is assessed. Then, determining if that level is normal or abnormal relative to the condition ids assessed. The therapeutic drug of interest is then administered, and the patient is then scanned again to determine if the DaT levels have changed (i.e., a follow-up scan). Both the baseline scan and the follow-up scan are accomplished following the steps below.

The patient has voided and is otherwise comfortable and prepared to lie still for the length of time required for xenon inhalation SPECT imaging using a SPECT camera with or without improved resolution capabilities (e.g., Discovery NM-630, GE Healthcare, Inc., Chicago, Ill. or inSPira HD®, Samsung Neurologica Corporation, Danvers, Mass.).

The subject is positioned in the camera and a peripheral 18 gauge to 22 gauge venous catheter inserted for the radiopharmaceutical infusion. A Y-system is used for optimal clearance of residual activity from the administration syringe.

The patient receives a single I.V. injection of [¹²³I]DAT2020 with a total activity dose amounting to 3.5 (±1.0 mCi.) The total administered radioactivity is of relevance and not the volume administered to achieve this dose. This single I.V. injection contains a maximum mass dose of DAT2020 of no more than about 16 ng and a total volume of up to about 5 mL. [¹²³I]DAT2020 must be administered manually via “slow I.V. injection”, followed by a 10 mL saline flush. As used herein “slow I.V. injection” refers to intravenous administration at about 5 ml/min to about 10 ml/min.

The exact radioactive dose administered is determined by calculating the difference between the radioactivity in the syringe and delivery system before and after injection. After the dose is delivered, the syringe is filled with a volume of saline equal to the administered dose volume. The syringe contents is recounted under the same conditions as used to determine the dose; separately. The delivery system is placed in a plastic container and counted in a dose calibrator (e.g., CRC®-25R Doe Calibrator, Capintec, Inc., Florham Park, N.J.) using the same parameters as used for the dose. Measured radioactivity values and times of measurement are documented in the source documents and recorded in the patient record, as well as the total injected volume. Injected radioactivity values outside the above stated range, i.e., values lower than about 7 mCi or higher than about 9 mCi are considered as potential sources of variation.

The single SPECT acquisition is commenced at 15 min (±2 min) post-injection for a 30 min scan. Specifically bound is required to determine striatal activity which demonstrates peak uptake during this scan time window. Thus, ensure the accuracy of the acquisition start time once the subject is injected with [¹²³I]DAT2020. The start and end times of the [¹²³I]DAT2020 scan are recorded on the imaging source document.

The acquisition parameters are recorded for each subject at the time of the scan on the imaging source document.

After preparing the dose as per the above, the patient is positioned in the SPECT camera as described above. Subjects are injected with 3.5±1.0 mCi (296 MBq) [¹²³I]DAT2020. The subject is positioned in the camera at the time of injection, even though imaging will not commence until 15 min (±2 min) after radiotracer infusion. [¹²³I]DAT2020 injection is administered by slow I.V. injection followed by a 10 mL saline flush. The start time of the injection is recorded along with the total volume injected.

Specific SPECT scan parameters, including collimation and acquisition mode, are set as follows. Raw projection data is acquired into a 128×128 matrix, stepping each head 3 degrees for a total of 120 projections into a 20% symmetric photopeak window centered on 159 keV for a total scan duration of approximately 30 min.

Tomographs of the captured photon data are then compiled and read by a radiologist skilled in assessing DaT levels using imaging.

Assessment of DaT levels in these patients prior to prescribing a drug regimen provides insight into the likely efficacy of the selected drug and hence allow for a patient-centric, individualized approach for a given patient. An initial scanning procedure to assess baseline DaT levels is conducted in the patient. The patient is then prescribed the selected drug therapy and periodic follow-up scans conducted for 2 to 3 subsequent months to assess DaT levels over time while on the drug. The results of these scans would be combined with the clinical observations of the patient by the physician to determine if the drug is having the desired biological and clinical effect.

### Equivalents

Those skilled in the art will recognize, or be able to ascertain, using no more than routine experimentation, numerous equivalents to the specific embodiments described specifically herein. Such equivalents are intended to be encompassed in the scope of the following claims.

What is claimed is:

1. An accelerated method of providing an active compound to a targeted region of the brain of a subject, served by the cerebral medial artery, the method comprising:
administering a sub-anesthetic dose of xenon as an inhalant to the subject;
and administering an efficacious amount of the active compound to the bloodstream of the subject,
the active compound reaching the targeted region of the brain in less time relative to a time it takes to reach the targeted region without xenon administration.

2. The method of claim 1, wherein the sub-anesthetic dose of xenon is from about 10% to about 43% xenon gas.

3. The method of claim 1, wherein the subject suffers from a disorder of the basal ganglia in the vascular bed of the middle cerebral artery.

4. The method of claim 3, wherein the disorder is a dopaminergic-related disorder.

5. The method of claim 1, wherein the active compound is administered intravenously to the patent.

6. The method of claim 1, wherein xenon is administered to the subject before, concomitantly with, or after the administration of the active compound.

7. The method of claim 1, wherein the active compound is an imaging or diagnostic agent.

8. The method of claim 7, wherein the imaging agent is a diagnostic imaging agent for SPECT, PET, or MRI.

9. The method of claim 7, wherein the imaging agent is DaTScan, DaT2020.

10. The method of claim 1, where in the active compound is a therapeutic compound for treating a dopaminergic disorder.

11. The method of claim 10, wherein the dopaminergic disorder is Parkinson's disease, attention deficit hyperactivity disorder, dementia, clinical depression, anxiety, narcolepsy, obesity, sexual dysfunction, schizophrenia, bipolar disorder, nausea/vomiting, addictive disorder (drugs, smoking), pheochromaocytoma, or binge eating disorder.

12. A method of evaluating an ability of a therapeutic composition to modulate a dopaminergic activity in the brain of a subject suspected of having a dopaminergic disorder, the method resulting in an accelerated delivery of the therapeutic composition across the blood brain barrier to a region of the brain served by the cerebral medial artery, the method comprising:
administering to the subject a sub-anesthetic amount of xenon gas as an inhalant at about 10% to about 43% before administering to the subject a radiolabeled tropane;
determining a baseline level and pattern of binding of the administered radiolabeled tropane to dopaminergic neurons in a portion of the brain of the subject;
treating the subject with an initial dose of a first therapeutic composition;
administering the radiolabeled tropane to the treated subject; and
determining the level and pattern of radiolabeled tropane binding to the portion of the brain of the treated subject,
a change in level and/or pattern of the radiolabeled tropane binding in the treated subject relative to baseline levels and/or patterns of radiolabeled tropane binding being indicative of the ability of the therapeutic composition to modulate a dopaminergic activity in the brain.
